(19) United States
(12) Patent Application Publication
(10) Pub. No.: US 2005/0059165 A9
(43) Pub. Date: Mar. 17, 2005

(54) Universal sample collection and testing system

(76) Inventors: James O. Davis, Riverside, CA (US); Brendan O'Farrell, Santa Ana, CA (US); Jeffrey S. Bauer, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/357,274

(22) Filed: Jan. 31, 2003

Related U.S. Application Data
(60) Provisional application No. 60/354,602, filed on Jan. 31, 2002.

(51) Int. Cl. G01N 33/558

(52) U.S. Cl. 436/514

(57) ABSTRACT

A sample collection and testing system comprises a collection device and a core device. The collection device comprises a main body and a number of interchangeable sampling apparatuses. The core device comprises a sample distributor and a number of independent testing strips that can simultaneously perform different tests on the same sample. Vents are provided to enhance wicking of sample through the test strips. In addition, a sample retention chamber or pocket is provided to maintain an unadulterated portion of the sample for subsequent retesting or confirmatory testing, for instance.

### BACKGROUND OF THE INVENTION

1. Field of the Invention

The present invention relates to a biological and/or environmental sample collection and testing system. More particularly, the present invention relates to a modular, integrated system of devices for collecting, treating, testing, and preserving biological and/or environmental samples.

2. Description of the Related Art

A wide variety of testing methods exist for the detection of analytes of interest. Simple field assays that can be performed by minimally trained personnel at the point of sample collection (“POS”) offer the advantages of convenience, faster test results, and reduced costs as compared to traditional centralized testing methods where tests are performed after a sample is collected and transported offsite to a centralized testing station or machine. Some exemplary technologies that allow for analyte detection tests to be done at point of sampling include, but are not limited to, biosensors, dry chemistry tests, rapid lateral-flow assays, and rapid flow-through assays.

The detection of analytes of interest has applications in many fields and disciplines, such as medical or veterinary diagnostics, environmental testing, testing of foodstuffs for quality, identity, contamination or adulteration, and the like. Nevertheless, in any application, the initial step when determining the presence of an analyte is collecting the sample that is to be tested.

Many samples require treatment before they can be tested. Treatment may involve mixing or diluting the sample in a buffer in order to correct analyte levels, dilute or remove interfering elements or contaminants, correct for adverse pH or ionic strengths, stabilize the analyte, extract the analyte in order to facilitate its detection, and the like. In some instances, the sample requires physical treatment to remove contaminants (e.g. microbes) or components that may interfere with testing (e.g. red blood cells in a blood sample or fat in a milk sample). In addition, some samples need to be concentrated in order to improve the performance of the assay, especially where the sample volume is small and/or the analyte concentration is low. Usually, the sample treatment step is performed before the assay, especially in the case of rapid assay methods, where sample collection and treatment add more time and steps to assay procedures that are preferably rapid and simple.

Additionally, sample collection and pretreatment can generate biohazardous waste. For example, when determining whether an analyte is present in saliva, a commonly used device for saliva collection is a collection pad from which the saliva is extracted using buffer extraction and/or physical separation under pressure. The treated sample is then added to a testing device, such as, for example, a lateral-flow test strip. The methods of sample collection and pretreatment described above require the use of a sample collection and a processing device. Furthermore, a separate testing device also must be used. Apparatus and materials used in each of these steps are potentially biohazardous and must be disposed of. The danger associated with biohazardous material can be even greater in the case of whole blood or contaminated environmental samples.

### SUMMARY OF THE INVENTION

Accordingly, a diagnostic system is desired that would allow the sample to be collected, treated and delivered to a substantially closed system for analysis, thereby minimizing the number of biohazardous byproducts generated by the collection, treatment, and testing methods. The system desirably would comprise all of the elements of the sample collection, processing and testing system so that all samples and their derivatives remain enclosed within a closed-system configuration once collection, treatment, delivery, and testing are in progress or completed, thereby protecting the device operators and the public from hazardous waste, such as that commonly encountered in the medical profession, for instance. Preferably, in some configurations, the device would include a method for inactivating any biohazardous material within the sample or device once testing is complete. For example, a system could utilize bactericidal or virucidal agents to treat the sample after the assay is complete.

In one configuration, an ideal diagnostic system would retain a portion of the sample for confirmatory testing, such as in the case of drug tests and HIV status testing. A system which retains and stabilizes, if necessary, a portion of the sample, such as saliva, would allow for later recovery of the sample for repeat testing. For example, retaining the sample would allow law enforcement agents and judicial officers to ensure the validity of a sample in a chain-of-custody situation.

Accordingly, one aspect of the present invention provides a modular, integrated system and method of using the components and/or devices of the system for collecting, treating, testing, and/or preserving samples of interest.

Another aspect of the present invention involves an easy to use, portable, multi-analyte, rapid diagnostic system comprising a closed-system configuration, which allows a user to obtain a variety of relatively fast test results with a single sample collection at the point of sampling. The system can be configured to reduce the risk of spreading or expelling any biohazardous material that may be present in and/or derived from the sample.

A further aspect of the present invention involves an integrated, diagnostic system comprising a closed-system configuration that can be used by an average person who is untrained in the use of diagnostic equipment.

Another aspect of the present invention involves a diagnostic system with a rugged construction and a closed-system configuration so that the system is liquid impermeable and operable under harsh weather and environmental conditions.

An aspect of the present invention also involves a universal sample collection device that can be adapted to collect a wide array of sample-types so that the system can detect the presence of multiple analytes from any number of sample sources depending upon the selected configuration.

A further aspect of the present invention involves a universal sample collection device with an indicator to verify that a sufficient volume of sample is collected and/or to determine whether the sample has been adulterated or is not the sample intended to be collected.

Another aspect of the present invention involves a device for treating and delivering a collected sample within a closed system so that a portion of the sample can be tested to determine the contents and/or properties of the sample, and so that there is little risk of releasing biohazardous material that may be present in the system.

A further aspect of the present invention involves a component for splitting a sample so that a portion of the sample is used for POS testing and another portion is preserved for offsite confirmatory testing.

Still another aspect of the present invention involves an integrated testing cassette capable of running multiple test formats, including but not limited to, rapid lateral-flow assays, rapid flow-through assays, and dry chemistry tests, in a simultaneous manner immediately following sample collection, treatment and delivery.

An aspect of the present invention also involves an integrated sample retention device in which a portion of the sample can be preserved for later or confirmatory testing.

An additional aspect of the present invention involves multiple retention chambers for preserving both tested primary samples and untested secondary samples of the same or different type, which makes it possible to conduct both confirmatory and complementary testing on multiple types of samples at a later time. For example, if the primary testing within the device is being performed with saliva, a whole blood sample may be taken and retained for confirmatory testing.

### BRIEF DESCRIPTION OF THE DRAWINGS

These and other features, aspects and advantages of the present invention will now be described with reference to the drawings of several preferred embodiments, which embodiments are intended to illustrate and not to limit the invention. The drawings comprise 13 drawings.

FIG. 1 is a side elevation view of a universal collection device arranged and configured in accordance with certain features, aspects and advantages of the present invention.

FIG. 2 is a perspective view of a core arranged and configured in accordance with certain features, aspects and advantages of the present invention.

FIG. 3 is a perspective view of another core arranged and configured in accordance with certain features, aspects and advantages of the present invention.

FIG. 4 is an enlarged exploded view of the core of FIG. 2.

FIG. 5 is an enlarged exploded view of the core of FIG. 3.

FIG. 6 is a section view of the core of FIG. 2 taken along the line 6-6.

FIG. 7 is a section view of the core of FIG. 3 taken along the line 7-7.

FIG. 8A is an exploded view of the universal collection device and core of FIGS. 1 and 2 with the universal collection device employing a swab.

FIG. 8B is an exploded view of the universal collection device and core of FIGS. 1 and 2 with the universal collection device employing a microcapillary tube.

FIG. 9A is an exploded view of the universal collection device and the core of FIGS. 1 and 3 with the universal collection device employing a swab.

FIG. 9B is an exploded view of the universal collection device and the core of FIGS. 1 and 3 with the universal collection device employing a microcapillary tube.

FIG. 10 is an enlarged section view of a portion of the core of FIG. 2 illustrating an exemplary placement of a lateral flow test strip.

FIG. 11 is a sectional view of an exemplary lateral flow test strip.

### DETAILED DESCRIPTION OF THE PREFERRED EMBODIMENTS

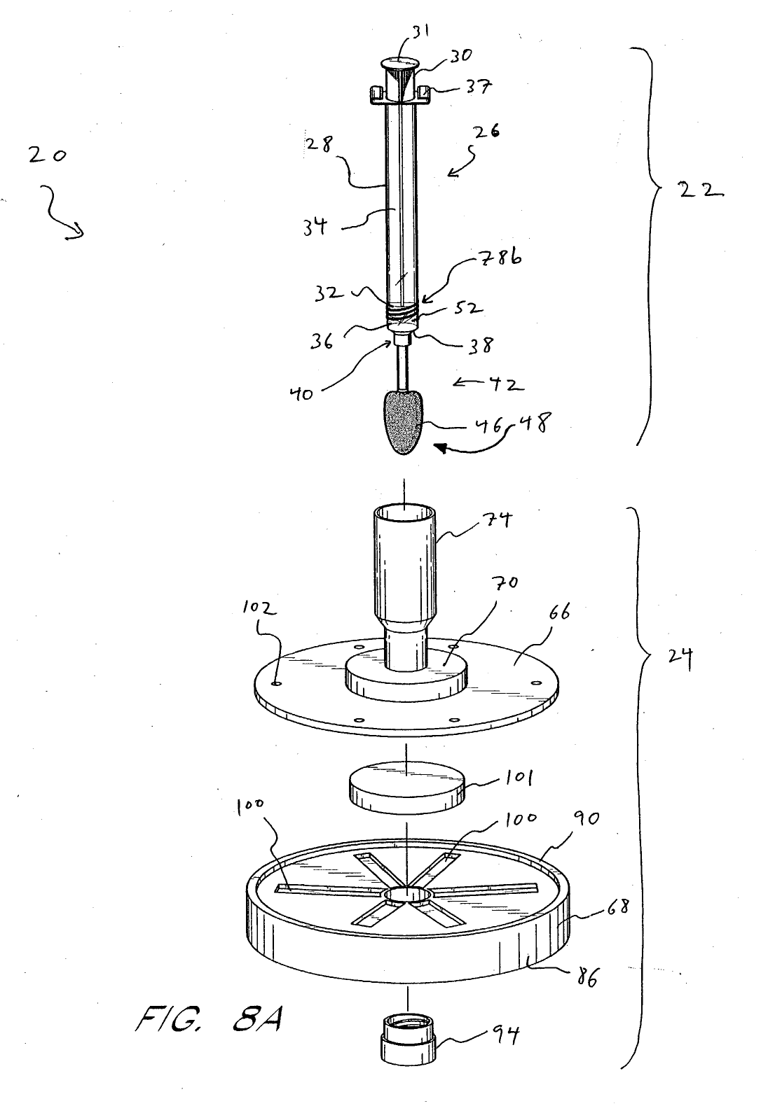

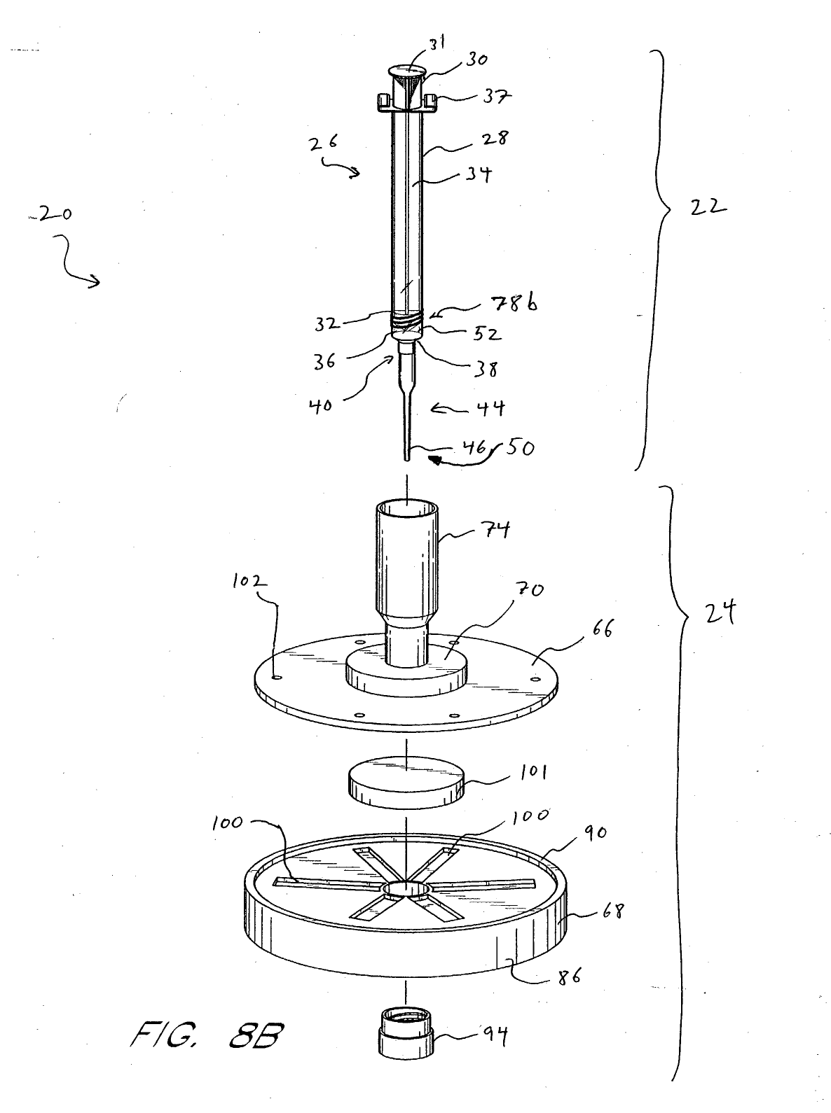

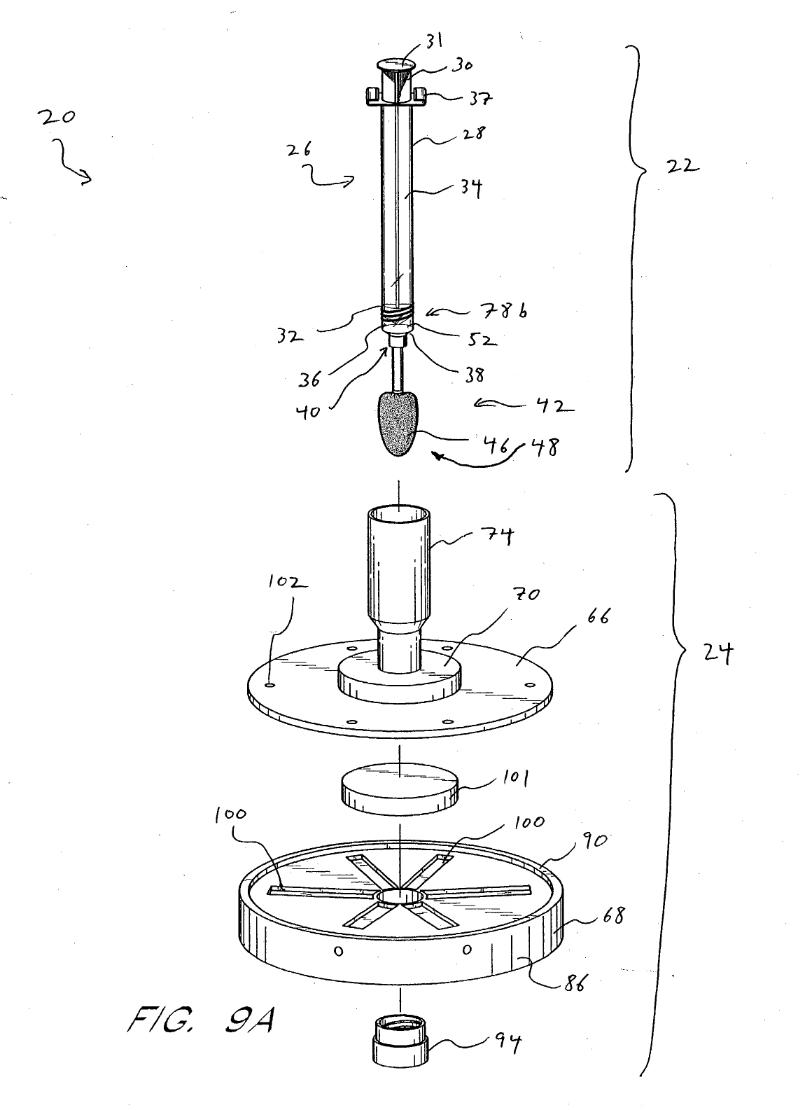

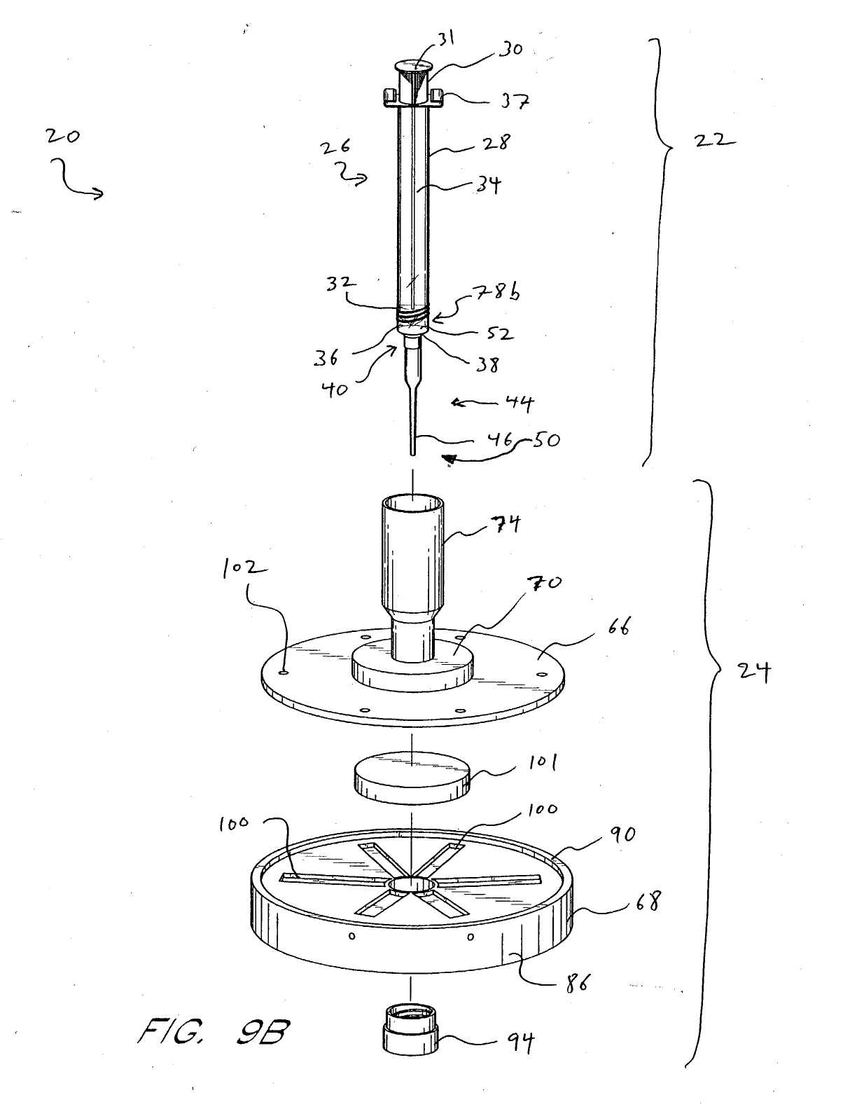

With reference to FIGS. 8A, 8B, 9A, and 9B, a few arrangements of a universal sample collection and testing system 20 are illustrated therein. In the illustrated configurations, the system 20 generally comprises two components: a universal sample collection device 22 and a core device 24. The universal sample collection device 22 and the core device 24 cooperate in manners that will be described. Prior to describing the interaction of the illustrated components 22, 24, however, each of the components will be described in detail.

Universal Sample Collection Device

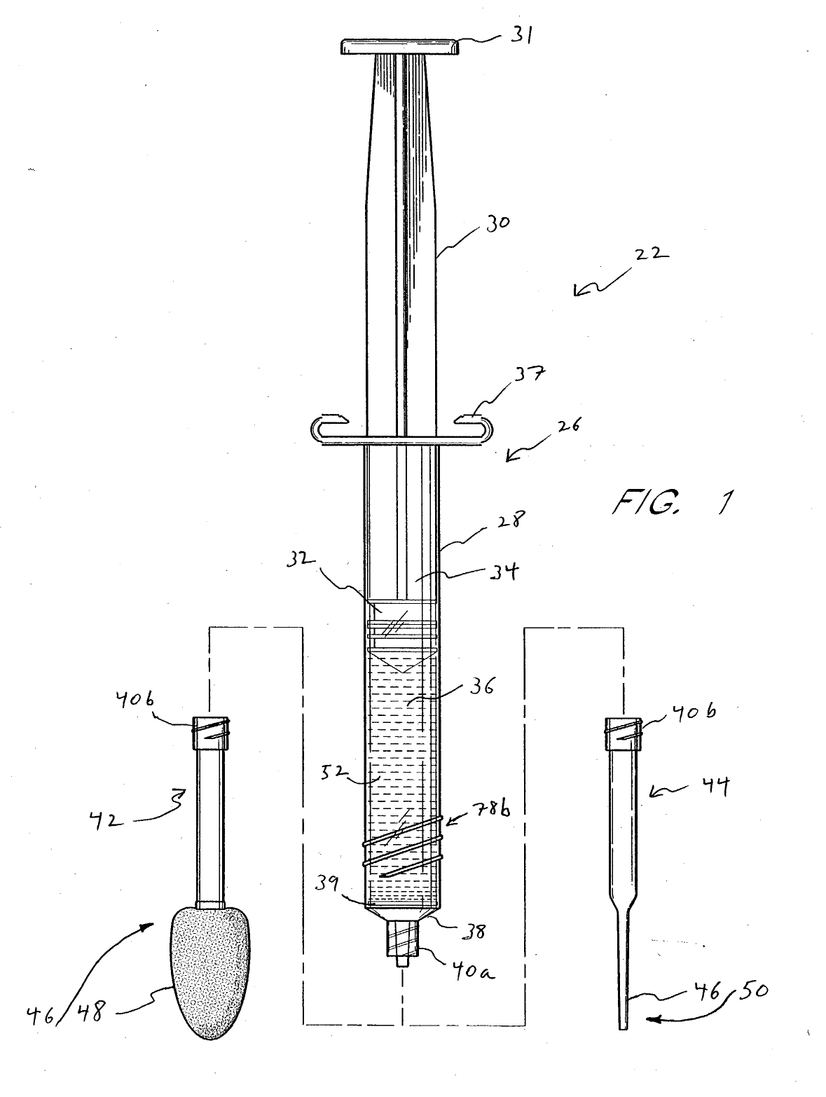

With reference now to FIG. 1, the universal sample collection device 22 generally comprises a main body 26. In the illustrated arrangement, the main body 26 is generally configured as any suitable industry standard syringe. The main body 26, in some arrangements, can comprise a tubular outer member 28 with a plunger body 30 that is disposed within the outer member 28.

The plunger body 30 defines a handle that extends into the outer member 28 in the illustrated arrangement. The illustrated plunger body 30 also comprises an integrated head 32 that divides the inside of the outer member 28 into an upper region 34 and a lower region 36.

An interface between the outer member 28 and the plunger body 30 preferably is sealed in any suitable manner such that movement of the plunger body upward in the illustrated arrangement draws a vacuum in the main body and movement of the plunger body downward in the illustrated arrangement forces the content of the lower region 36 out of the main body 26.

In one arrangement, the main body 26 can comprise a snap-lock 37 such that the plunger body 30 is secured in position once the head 32 has been depressed to a desired extend. Preferably, the snap-lock 37 secures the plunger body 30 in position once the contents of the lower region 36 have been forced out of the main body 26 by the plunger body 30. In the illustrated arrangement, the snap-lock 37 is positioned at an upper end of the main body and secures the proximal end (i.e., the upper end in the illustrated arrangements) 31 of the plunger body 30 in position. In one embodiment, the snap-lock 37 utilizes a spring-biased member or design to secure the top end 31 in position. In other arrangements, the snap-lock 37 may be disposed internally within the main body, even at a lower position within the lower region 36.

A distal end (i.e., the lower end in the illustrated arrangements) of the outer member 28 preferably tapers to a nozzle 38. In one arrangement, the nozzle 38 comprises a pressure-breakable seal 39 such that at least the lower region 36 is sealed to provide security against contamination of any sample drawn into the lower region 36, whether on purpose or inadvertently. The seal 39 also guards against undesired leakage during storage or shipping prior to use, as will be described.

The nozzle 38 also can comprise a portion 40a of a universal coupling 40. The coupling 40 defines a connection point for various sample collection components, such as fixture heads 42, 44, described below. Thus, the coupling 40 preferably allows for universal connection of a variety of sample collection components. In one presently preferred arrangement, the coupling 40 is configured similar to a luer-type fitting, which allows rapid exchange of components through simple twist actions. In other arrangements, the coupling 40 employs unique constructions that will limit the availability of certain components for use with the collection device 22 such that users have a type of fail-safe mechanism for determining which fixture heads 42, 44 should be used with their collection device 22. For instance, in one embodiment, a main body 26 having a three lugged (or three thread) construction would not be able to be used with a fixture head having a two lug (or two thread) receiving construction.

The collection device 22 also comprises the fixture heads 42, 44 introduced above. Any suitable fixture head can be used and the illustrated fixture heads 42, 44 are but two examples of sample collection components that are adapted for use with the collection device 22. As illustrated, each of the fixture heads 42, 44 comprise a portion of the universal coupling 40b on one end and a collecting apparatus 46 on the opposing end. Thus, portion 40a and portion 40b preferably can be rapidly connected together and can be rapidly separated. As discussed above, the coupling 40 can be constructed to limit the use of certain fixture heads with certain main bodies and vice versa.

The collecting apparatus 46 disposed along the fixture head 42 and/or defined by the fixture head 42 can vary widely. For example, as illustrated in FIG. 1, the collecting apparatus 46 may be an absorbent swab 48 or a microcapillary tube 50. The collecting apparatus 46 can be any of a number of suitable absorbent devices that are adapted to collect or extract the desired sample. Examples of collecting apparatuses include, but are not limited to, pads, nibs, capillary tubes, filter paper, swabs, and the like (and combinations thereof).

The collecting apparatus 46 generally can be used by inserting, dabbing or swiping the collecting apparatus onto or through the desired sample source. In addition, the sample can be withdrawn into the main body 26 through the collecting apparatus 46 by forming a vacuum in the lower region 36 of the main body 26. Furthermore, in yet other arrangements, the collecting apparatus 46 is not attached to the main body 26 during collection but is detached during collection; the collected sample then is transferred into a region that allows interaction with the balance of the system 20 in manners described herein.

The type of head fixture 42, 44 selected for use with the sample collection device 22 generally depends upon the type of sample being collected. For example, if the desired sample were blood or serum, the head fixture 44 could be a capillary device, such as the microcapillary tube 50 illustrated in FIG. 1. If the sample source were milk, water, processed food, or fecal matter, an appropriate head fixture 42 could include a suitable filtering device (not shown) to remove particulates as a volume of sample is drawn into the main body 26. If the collection device 22 were to be used to collect saliva samples, an appropriate head fixture 42 could be an absorbent pad (not shown) capable of absorbing a defined volume of fluid. The pad (not shown) could be an absorbent paper, foam, or other material.

In some arrangements, an indicator (not shown) may be included in either the head fixture 42, 44 or in the main body 26 to verify when a sufficient testable volume of a sample (such as saliva, for example) has been collected and/or to indicate whether the sample is incorrect or has been adulterated since drawing.

In one preferred embodiment, the lower region 36 of the main body 26 is filled with a buffer solution 52. In one particularly preferred arrangement, the buffer solution 52 prefills the lower region 36 of the main body 26 (e.g., it is placed there during manufacture of the collection device 22). In such an arrangement, the seal 39 reduces the likelihood of unintended loss of buffer solution 52. In addition, the seal 39 can maintain a separation between the head fixture 42 and the buffer solution 52 until contact of the two is desired. As such, the seal 39 can be disposed within the head fixture, upstream of the collecting apparatus 46 (or an additional seal can be provided).

The buffer solution 52 can act as a sample diluent and/or a sample stabilizer. When acting as a sample stabilizer, the sample can be stored for extended periods of time at room or refrigeration temperatures. In one arrangement, the buffer solution 52 can be a running buffer for the assay performed in the test device 20. In other arrangements, the buffer solution 52 can function as a processing or stabilization buffer for the desired test sample.

The buffer solution 52 generally aids in the expulsion of the sample from the head fixture 42, 44 attached to the collection device 22. In one particularly preferred arrangement, the buffer solution 52 does not contact the head fixture 42, 44 until the plunger body 30 is depressed, thereby forcing the buffer solution 52 under pressure through the breakable seal 39 that precedes the collecting apparatus 22 (and the coupling 40 in some arrangements) and into the head fixture 42, 44. In some other arrangements, however, sample is drawn into the lower region 36 of the main body 26 prior to expulsion. In such arrangements, the seal 39 can first be broken and then the sample can be drawn into the main body 26.

Core Device

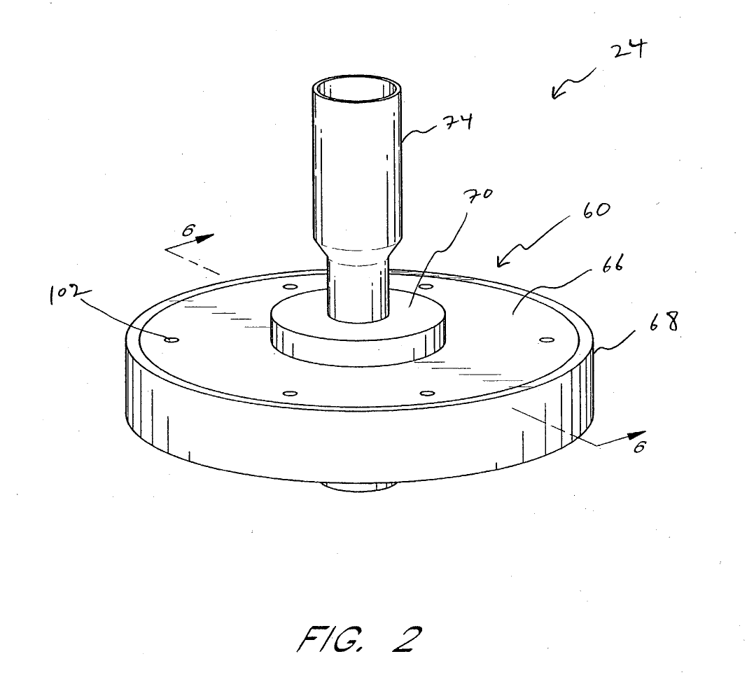

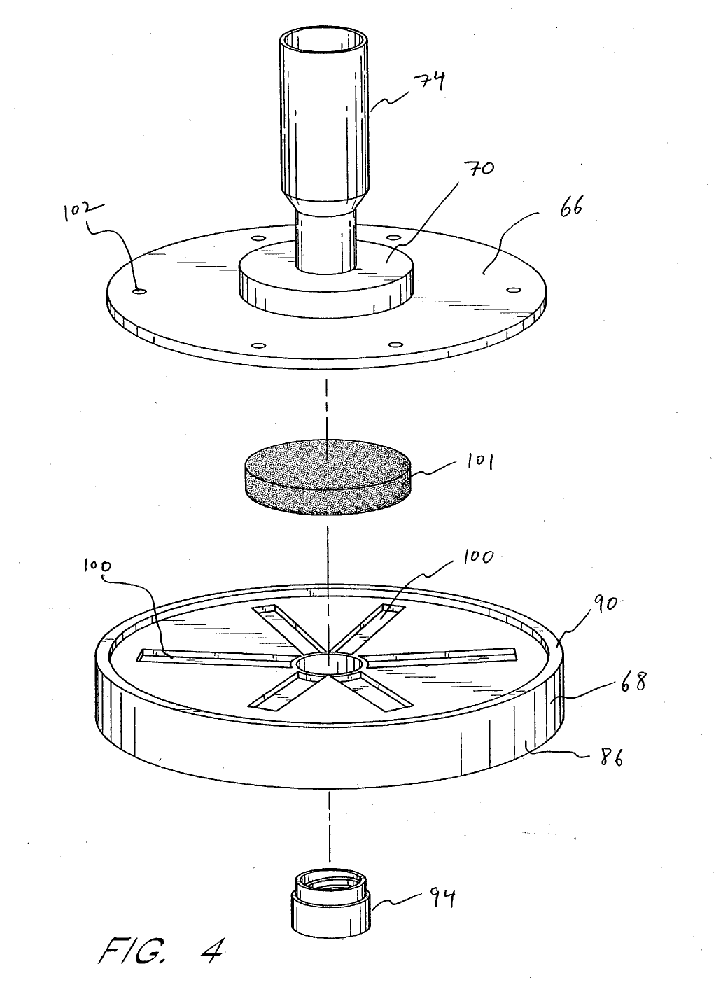

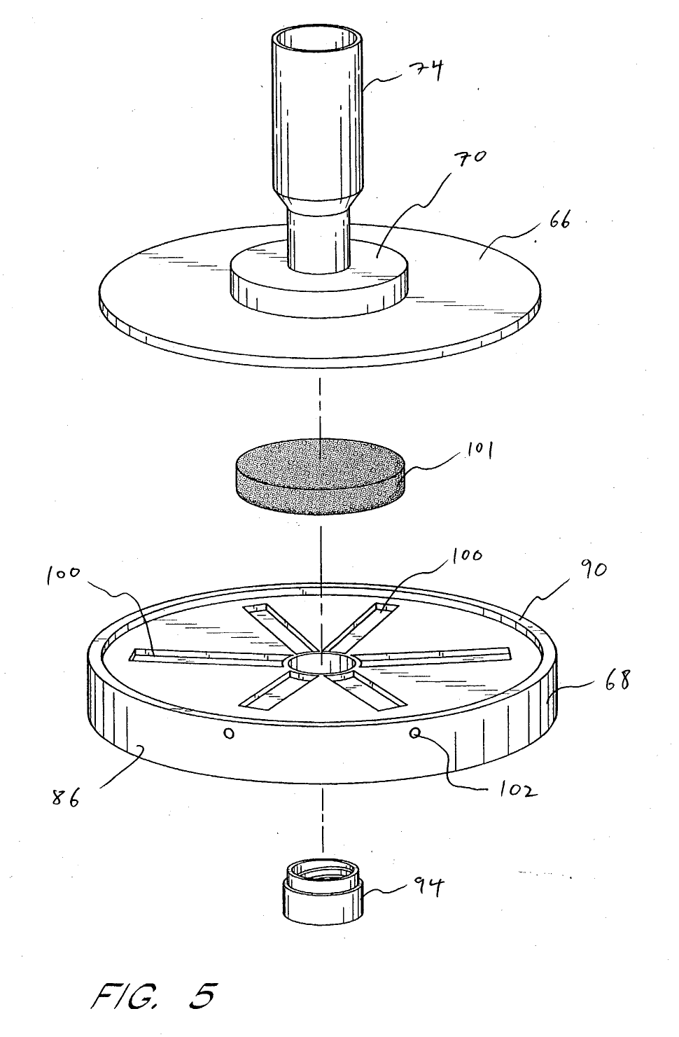

With reference now to FIGS. 2-7, a first arrangement of the core device 24 arranged and configured in accordance with certain features, aspects and advantages of the present invention is illustrated. The illustrated core device 24 generally comprises an outer housing 60.

The outer housing 60 comprises a generally disc-shaped structure in the illustrated arrangement. The disc-shaped portion of the illustrated housing 60 defines a cassette. It is anticipated that other housing configurations and cassette configurations also can be used. However, the generally disc-shaped construction results in a relatively compact construction. In one presently preferred configuration, the disc-shaped construction has a diameter of about 140 mm and a thickness of about 5 mm. Other sizes can be used depending upon the application. For instance, a system 20 designed for high numbers of simultaneous tests will likely be larger in diameter.

The housing in the illustrated arrangement comprises an upper member 66 and a lower member 68. The upper member 66 and the lower member 68 can be secured together in any suitable manner. In one preferred arrangement, the members 66, 68 are formed of a thermoplastic material and are ultrasonically welded together. In other arrangements, the members 66, 68 can be secured using any other suitable technique, including but not limited to mechanical interlocks, snap-fits, glue, other methods of adhesion and cohesion (e.g., ultraviolet curable glue) and the like. In the presently preferred arrangement, the housing 60 is formed of a clear and transparent plastic material suitable for molding, such as, for example, polycarbonate and derivations/combinations thereof.

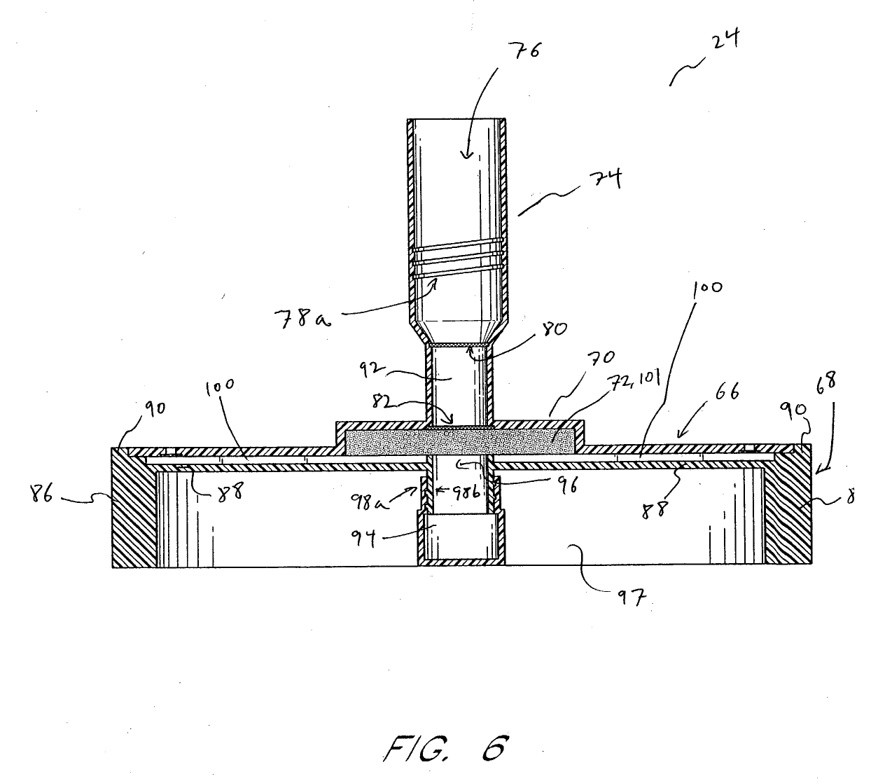

With reference now to FIG. 6, the illustrated upper member 66 comprises an upset central region 70. The upset central region 70 preferably defines a centrally located recess 72. In the illustrated arrangement, the centrally located recess 72 is concentrically located such that it is centered along a central axis of the cylindrical portion of the housing.

Additionally, the upper member 66 comprises a connection port 74. The connection port 74 in the illustrated arrangement extends upward from the cylindrical portion of the housing 60 and, more particularly, from the upset region 70.

Preferably, the connection port 74 defines a connecting lumen 76 that extends downward into the cylindrical portion of the housing 60. The connecting lumen 76 desirably is sized and configured to mate with the nozzle 38 and a portion of the main body 26 of the collection device 22. More preferably, an interior surface of the connecting lumen 76 is provided with a portion 78a of a secondary coupling 78, which also features another portion 78b that is disposed along an outer surface of the main body 26. In the illustrated arrangement, the secondary coupling 78 is a luer-type of connection. Again, as with the first coupling 40, any suitable coupling configuration can be used.

The main body 26 and the connection port 74 can be joined together with the secondary coupling 78. As such, the secondary coupling 78 advantageously defines a locking mechanism between the housing 60 of the core device 24 and the sample collection device 22 in one embodiment. This locking mechanism advantageously seals the system 20 and encapsulates all assay reagents and samples inside the system 20 once the core device 24 and the sample collection device 22 are connected. In some arrangements, the locking mechanism can be configured to permanently or semipermanently lock the two components together to greatly reduce the likelihood that the two components can be separated once connected together.

With continued reference now to FIG. 6, a particulate filter 80 can be located within a distal section of the connecting lumen 76 and a second particulate filter 82 can be located proximate an intersection of the connecting lumen 76 and the upset region 70. Thus, the particulate filters 80, 82 can be disposed adjacent to an end of the connecting port 74 and/or within the connection lumen defined within the connecting port 74. The particulate filters preferably are made of a porous organic or inorganic material, such as HDPE, borosilicate glass, ceramic material, and the like. In other arrangements, the core device 24 has only one filter, either in the connection lumen 76 or elsewhere in the housing 60 while, in some arrangements, no filter is used at all. In yet another arrangement, the core device 24 comprises an assembly of three or more tiers of particulate filters.

With continued reference to FIG. 6, the lower member 68 preferably comprises an outer wall 86 and a second wall 88 that extends generally transverse to the outer wall 86. In the illustrated arrangement, the outer wall 86 is generally cylindrical in shape with the second wall 88 being disc-shaped and extending across substantially the entire diameter defined by the outer wall 86. Again, the actual shape of the housing 60 and its components and members, such as, for example, members 66, 68, can be varied.

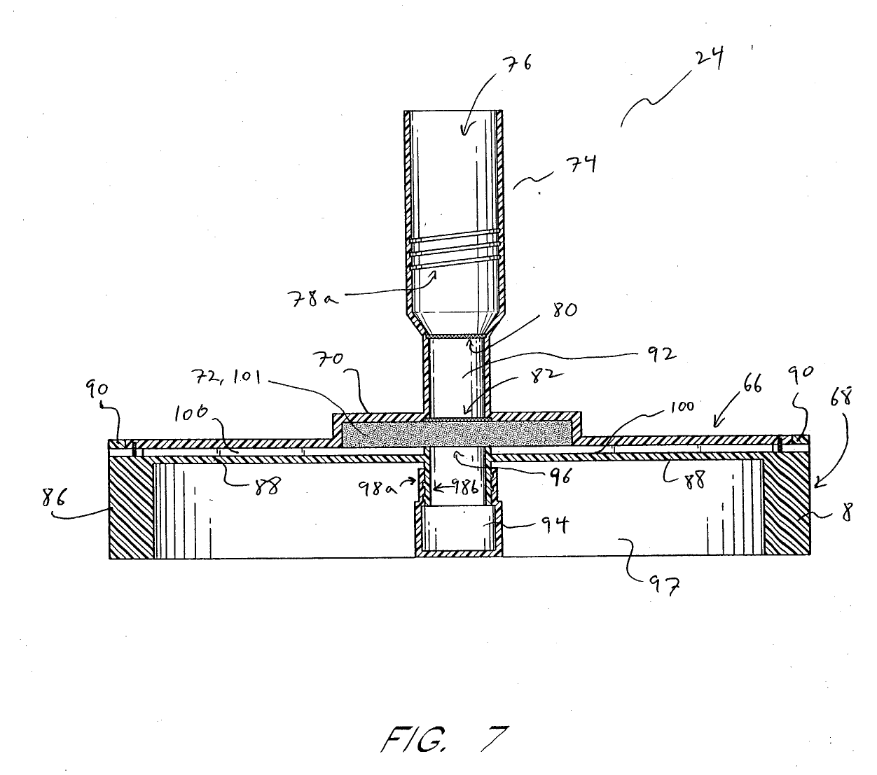

The second wall 88 preferably is inset from both axial ends of the outer wall 86 such that a recess can be defined on each side of the wall 88. Additionally, the upper end of the outer wall 86 preferably is provided with a step 90 that receives the upper member 66 to provide a more secure connection between the upper member 66 and the lower member 68. Together, the upper member 66 generally, the lower end of the connecting lumen 76, and the upper end of region 70 preferably define a core chamber or a core lumen 92. In one embodiment, the core lumen 92 extends from the connecting lumen 76 and extends within a fairly large portion of the core device 24. With reference to the embodiment illustrated in FIGS. 6 and 7, the upper member 66, the filter 80 and the filter 82 preferably define a core chamber or a core lumen 92.

Preferably, the core device 24 comprises a sample retention chamber or pocket, which, in one embodiment, is a sterile, closed vial or vessel. The presence of a retention chamber facilitates the recovery and preservation of samples for later testing or forensics evidence, for instance. The lower member 68 of the housing 60 preferably comprises a centrally located aperture 96 that extends downward from the second wall 88. This aperture 96 can be cylindrical in some arrangements. A lower end 98b of the aperture 96 preferably mates with a wall 98a of the separable retention chamber member 94. Preferably, the chamber member 94 is sized to be contained within a recess 97 defined by the outer wall 86 and the second wall 88.

The chamber member 94 and the housing 60 can be secured together with a mechanically interlocking structure 98, such as a luer-type construction, for instance. The interlocking structure preferably reduces the likelihood of sample leakage when connected. Thus, when the retention chamber member 94 is attached, an air-tight and liquid-tight seal preferably is formed between the housing 60 and the retention chamber member 94. Advantageously, the chamber member 94 preferably is positioned at a lowermost point of the core device 24 such that any excess sample remaining the in other portions of the core device can fall into the chamber member 94 under the forces of gravity.

In some arrangements, a highly absorbent material (not shown), which can be pre-treated to promote analyte stability over time, is positioned within the retention chamber member 94 to facilitate the transfer of excess sample into the retention chamber member 94. This material also can facilitate the retention and stabilization of the sample within the retention chamber member 94. The highly absorbent material in the retention chamber member 94 can comprise numerous materials, such as hydrogel, absorbent paper, sponge-like materials with high saturation characteristics, and the like, and combinations thereof. Nevertheless, some of the sample may be retained in the cassette components, which will be discussed below.

In another arrangement, the core device 24 can comprise a second retention chamber (not shown). The second retention chamber (not shown) can be a sterile, closed vial or another vessel that holds a secondary or complementary sample. For example, in one configuration, the second retention chamber (not shown) can be designed to hold a blood sample from a medical patient in order to accompany saliva samples that are tested and retained in a first retention chamber member 94. The second retention chamber (not shown), if used for blood, preferably contains an anti-coagulant such as, for example, heparin, in order to prevent clotting of the blood within the chamber. The preservation of a secondary blood sample to accompany the primary saliva sample within the system described above has numerous benefits, including a more complete profile of a patient, an additional type of sample (i.e. blood) with which to corroborate or reject any data or evidence based on the first type of sample (i.e. saliva), and more sample(s) in general with which to run later tests. In one preferred embodiment, the second retention chamber (not shown) contains an absorbent material known in the art to stabilize the analyte of interest or other components within the sample and/or to allow for the extraction of the analyte when confirmatory testing is to be performed. Other numbers of retention chambers (e.g. more than two) also can be used.

The second wall 88 also preferably comprises a number of integrally formed grooves 100 that extend though the core chamber 92. The grooves 100 in the illustrated arrangement extend away from but do not intersect with the central aperture 96. In other words, the grooves 100 originate slightly outwardly from the outer circumference of the central aperture 96 in the illustrated arrangement.

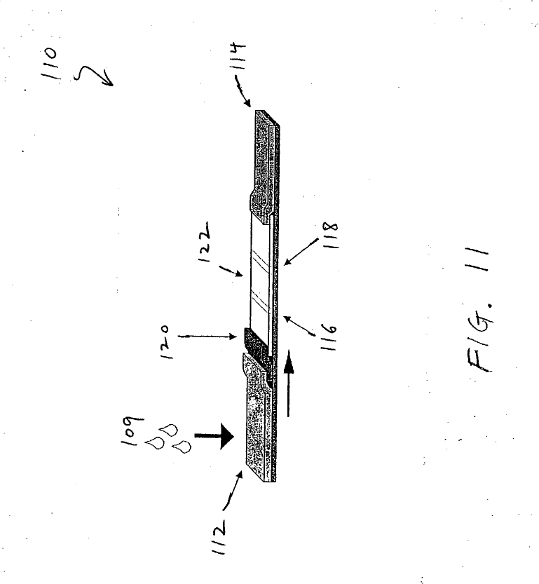

The grooves 100 preferably are fairly shallow and narrow. Desirably, the grooves 100 are sized to fit conventional lateral flow test strips, but the grooves 100 may be sized or configured to fit different testing devices. Thus, the grooves 100 are designed to receive testing devices, such as, for example, biosensors, dry chemistry tests, rapid lateral-flow assays, rapid flow-through assays, etc. For instance, the testing devices can be standard testing strips that are known in the art and used to detect the presence of certain analytes. In a preferred embodiment, each test strip runs a separate rapid-lateral-flow or rapid-flow-through assay (“rapid assay”). FIG. 11 illustrates a standard lateral-flow test strip 110. Lateral-flow test strips 110 typically comprise a sample application pad 112, a wick 114, a test line 116, and a control line 118. When the sample 109 is applied to the application pad 112 on one end of the strip 110, the wick 112 draws the sample toward to other end of the strip 110, thereby causing the sample to move across both the test line 116 and the control line 118. In one embodiment, a positive test line indicates the presence of a certain substance, compound, material, etc., and a positive control indicates that the result displayed on the test line is reliable. In one embodiment, illustrated in FIG. 11, the test strip 1 10 comprises a conjugate release pad 120 and membrane substrate 122.

The number of grooves 100 varies depending on the number of tests or assays desired. There are six grooves 100 in the illustrated arrangements. The grooves 100 extend outward from a central axis and preferably are spaced evenly apart. The angle of separation between any two adjacent grooves 100 preferably is 360°/n, where n equals the number of grooves 100 that extend outward from the well. Therefore, in the illustrated arrangements, the angle of separation between any two adjacent slots is 60°. The slots also can be spaced assymetrically if desired.

With reference again to FIG. 6, a sample distributor 101, and the recess 72 in which it fits, preferably are sized so that the outside, lower portion of the sample distributor 101 is in fluid communication with a sample application portion of each of the testing devices (e.g. test strips). More preferably, the sample distributor 101 is centrally located relative to each of the grooves 100. In the illustrated arrangement, at least a portion of the sample distributor 101 overlies a portion of each of the test strips and/or grooves 100. As such, the illustrated sample distributor 101 is located above the test strips. In some arrangements, the sample distributor 101 can be disposed adjacent to (i.e., abutting) or can underlie each of the test strips and/or grooves 100. In a presently preferred arrangement, the sample distributor 101 preferably is configured such that any excess sample that remains in the sample distributor 101 at the conclusion of the rapid assays get transferred into the retention chamber member 94.

In one arrangement, the sample distributor 101 can be an absorbent pad with low saturation characteristics. The absorbent pad may be composed of numerous types of material, such as paper, sponge, etc. Preferably, the absorbent pad absorbs fluids and distributes moisture within the pad in a generally even manner. In arrangements featuring a sample distributor 101 with low saturation properties, the sample distributor and the housing preferably are configured such that most of the remaining sample drips into the retention chamber member 94. In another arrangement, the sample distributor 101 may be configured as hydrophobic and hydrophilic coatings, microfluidic channels, and the like that act to provide adequate supplies of sample from a central portion of the core device 24 to each of the test strips and/or grooves 100.

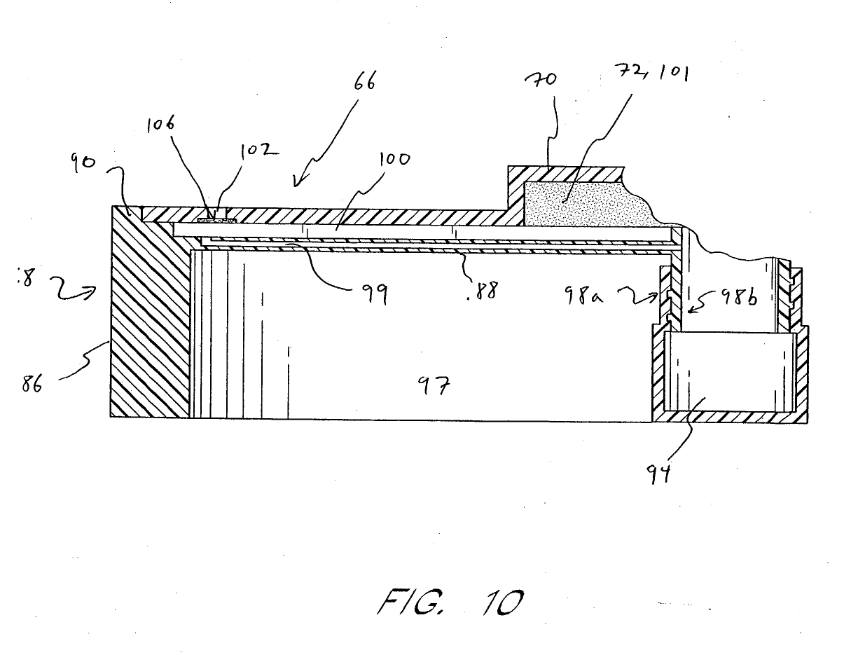

In one arrangement, the housing 60 can comprise runoff conduits 99 (see FIG. 10) that connect the outer portions of the grooves 100 to the retention chamber member 94, thereby channeling any runoff or excess sample from the lateral-flow test strips into the retention chamber member 94. In yet another arrangement, one of the grooves 100 can receive a “dummy” test strip that serves as a retention chamber.

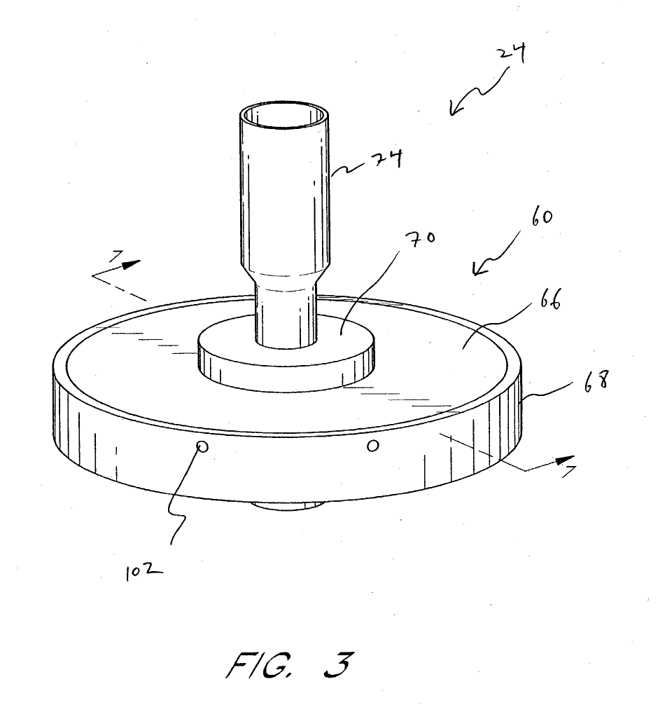

With reference now to FIGS. 2, 3, and 10, a number of ventilation ports 102 preferably are defined in the housing 60. In the illustrated arrangements, six ventilation ports are shown. The number of the ventilation ports can be increased or decreased depending upon the application. As illustrated, the ventilation ports 102 can extend through the upper member 66 (i.e., extend vertically in the illustrated arrangement—See FIG. 2) or can extend through the side outer wall 86 (i.e., extend horizontally in the illustrated arrangement—See FIG. 3).

The location of the ventilation ports 102 can be determined by the location and distribution of the testing devices and/or the pre-molded slots for the testing devices. Desirably, the ventilation ports 102 are positioned along at least a portion of the test strips and/or grooves 100. In one particularly preferred arrangement, the ventilation ports 102 are positioned at the outer end of the test strips and/or grooves 100 to better facilitate wicking of sample into and along the test strips. In another arrangement, the ventilation ports 102 can be positioned on the upper member 66 so that each port 102 is equidistant from any adjacent port 102, the center of the upper member 66, and the outer edge of the upper member 66.

With reference now to FIG. 10, each ventilation port 102 preferably is filled and/or covered with a microporous filtering material 106 that is permeable to gas but impermeable to liquid. Examples of appropriate microporous material for the ventilation ports include materials sold under the trademarks of Tyvek, Gortex, and the like. In some arrangements, the ports 102 can be sized to limit, or prevent fluid flow through the ports 102.

In use, the collecting device 22 is inserted into the connection port 74 of the core device 24. The main body 26 of the collecting device 22 preferably locks into place within the connecting lumen 76, thereby forming an air-tight and fluid-tight seal between the collection device 22 and the core device 24. For example, in the case of saliva samples, after the sample has been collected by inserting the handheld device into the subject's mouth for a given period, the entire collection device 22 can be locked into the connecting lumen 76 of the core device 24, thereby sealing the system 20.

In one arrangement, locking the collecting device 22 with the housing 60 of the core device 24 effects both sealing of the system 20 and the compression of the pad of the head fixture 42, which contains the sample, against the filter 80 located within the connecting lumen 76. Physical compression of the sample containing head fixture 42 in the manner just described is particularly useful if the main body 26 does not contain a buffer solution with which to flush the sample out of the pad.

After connecting the collection and core devices 22, 24, the plunger body can be depressed to force the sample contents out from the head fixture 42 and into the core chamber 92 in which the distributor 101 is positioned. In one particularly preferred arrangement, the plunger head 32 locks into a snap-lock mechanism located at the end of the main body 26 proximate the nozzle 38. In an arrangement featuring a buffer solution 52, the act of depressing the plunger body 28 forces buffer fluid 52 into the head fixture 42. Thus, the buffer solution 52 mixes with the collected sample and the mixture is flushed out of the head fixture 42 into the core chamber 92 that contains the sample distributor 101. In another arrangement not containing buffer solution 52, the act of depressing the plunger body 28 increases the air pressure within the lower region 36 and the escaping air forces the collected sample to move into the core chamber 92 that contains the sample distributor 101. As described above, any number of filters can intercede between the collection device 22 and the sample distributor 101.

After the sample reaches the distributor 101, the sample is distributed to the sample application pads of the testing devices. The user then waits a set amount of time to observe the results of the rapid assays. The user will know the results of the various rapid assays by observing physical indicators on the testing devices. In one embodiment, the physical indicator for the presence of a certain analyte is a distinctly colored band on a section of a test strip observable through the clear and transparent structure which houses the test strips. In another embodiment, distinctly colored bands indicate the absence of certain analytes or indicate that the rapid assay is done. In yet another embodiment, the test results are transduced as electrical potential or resistance values, the strength of a magnetic field, the optical density of a visible signal, and/or the strength of a fluorescent signal. Here, a companion reading system is used to detect and interpret the transduced signal values.

Although certain preferred embodiments and examples are disclosed above, it will be understood by those skilled in the art that the scope of the present invention extends beyond the specifically disclosed embodiments to other alternative embodiments and/or uses of the invention and obvious modifications and equivalents thereof. For instance, certain features, aspects and advantages of the present invention can be used with medical apparatus, such as whole blood machines and the like. Thus, it is intended that the scope of the invention herein disclosed should not be limited by the particular disclosed embodiments described above. In addition, certain features, aspects and advantages of any one embodiment can be used in other embodiments and, as such, the several embodiments are capable of various combinations.

1. An integrated collection and testing system, said system comprising a sample collection device and a core device, said sample collection device and said core device being separated during collection and integrated during testing, said core device and said collection device being permanently locked together when integrated after sampling.

2. The system of claim 1, wherein said core device comprises a plurality of test strips, at least two of said plurality of test strips designed to test for a different analyte.

3. The system of claim 2, wherein said core device comprises a vent positioned proximate each of said test strips.

4. The system of claim 3, wherein said vent comprises a water-impermeable but air-permeable covering.

5. The system of claim 3, wherein said core device comprises a sample distributor that is in fluid communication with said test strips.

6. The system of claim 5, wherein said core device further comprises an additional vent positioned proximate said distributor.

7. The system of claim 6, wherein said additional vent comprises a water-impermeable but air-permeable covering.

8. The system of claim 6, wherein said core device is in fluid communication with a sample retention chamber.

9. The system of claim 8, wherein excess sample passes from said sample distributor to said sample retention chamber.

10. The system of claim 1, wherein said core device comprises a sample retention chamber.

11. The system of claim 1, wherein said sample collection device is inhibited from multiple sample collection with a plunger body snap-lock.

12. A universal collection and testing system, said system comprising a sample collection device, said device comprising a main body and a plurality of interchangeable fixture heads, each of said plurality of interchangeable fixture heads comprising a different collection member, said main body and each of said plurality of interchangeable fixture heads comprising a portion of an interlocking structure configured to mate said main body with any one of said plurality of interchangeable fixture heads.

13. The system of claim 12, wherein said main body comprises a syringe.

14. The system of claim 13, wherein said syringe comprises a fluid-retaining portion, a nozzle and a pressure-breakable seal disposed between said nozzle and said fluid-retaining portion.

15. The system of claim 14, wherein said seal is interposed between said fluid-retaining portion and a selected one of said plurality of interchangeable fixture heads when said selected fixture head is coupled with said syringe.

16. The system of claim 13, wherein said syringe is filled with a buffer solution.

17. The system of claim 12 further comprising a core device, said core device comprising a first portion of an interlocking structure and said collecting device comprising a second portion of said interlocking structure, said interlocking structure adapted to provide an air-tight and liquid-tight seal between said core device and said collecting device.

18. The system of claim 17, wherein said interlocking structure comprises a luer-type connection.

19. A universal collection and testing system, said system comprising a sample collection device and a core device;
said collection device comprising a main body and one or more interchangeable fixture heads, each of said fixture heads comprising a different collection member;
said core device comprising one or more testing devices and one or more sample retention chamber member.

20. The system of claim 19, wherein said main body comprises a syringe.

21. The system of claim 20, wherein said syringe comprises a fluid-retaining portion, a nozzle and a pressure-breakable seal disposed between said nozzle and said fluid-retaining portion.

22. The system of claim 21, wherein said syringe is filled with a buffer solution.

23. The system of claim 19, wherein said core device comprising a first portion of an interlocking structure and said collecting device comprising a second portion of said interlocking structure, said interlocking structure adapted to provide an air-tight and liquid-tight seal between said core device and said collecting device.

24. The system of claim 23, wherein said interlocking structure comprises a luer-type connection.

25. The system of claim 19, wherein said testing device comprises a lateral flow test strip.

26. The system of claim 19, wherein said one or more sample retention chamber member comprises a dummy test strip.
